(19) United States
(12) Patent Application Publication
(10) Pub. No.: US 2025/0135093 A1
(43) Pub. Date: May 1, 2025

(54) Method and System for Wound Monitoring and Pathogen Detection

(71) Applicant: Tao Treasures LLC DBA Nanobiofab, Frederick, MD (US)

(72) Inventors: Xiaonao LIU, Frederick, MD (US); Taejun KO, Frederick, MD (US); Deyu LIU, Frederick, MD (US); Derese GETNET, Frederick, MD (US)

(21) Appl. No.: 18/933,674

(22) Filed: Oct. 31, 2024

Related U.S. Application Data
(60) Provisional application No. 63/594,900, filed on Oct. 31, 2023.

(51) Int. Cl. A61M 1/00; A61F 13/00; A61F 13/05

(52) U.S. Cl. CPC A61M 1/95; A61F 13/05; A61F 2013/00965; A61M 2205/3592; A61M 2205/502

(57) ABSTRACT

A system for wound monitoring and pathogen detection includes a sensor device having an inlet connected to a NPTW dressing and an outlet connected to a NPTW pump. The sensor device includes a sensor array configured to detect a gaseous emission from one or more pathogens present in a wound by generating signals upon exposure to one or more compounds in the gas phase; a microcontroller (MCU) operatively coupled with the sensor array, the MCU configured to process signals from the sensor array to identify the one or more compounds; a wireless communication component for transmitting data from the MCU to one or more external devices; and one or more visual indicators that presents information comprising an operational mode of the sensor device and a presence or absence of pathogen in the gaseous emission. A method for wound monitoring and pathogen detection is also provided.

### CROSS-REFERENCE TO RELATED APPLICATIONS

This application claims the benefit of priority under 35 U.S.C. § 119 to U.S. Provisional Application No. 63/594,900, filed Oct. 31, 2023, the entire contents of which are incorporated herein by reference in their entireties.

### TECHNICAL FIELD

The present invention generally relates to a pathogen detection and wound infection monitoring system for advance wound care, specifically a wound monitoring system that employs a sensor device having a sensor array and operates at a reduced pressure.

### BACKGROUND

Timely detection of pathogens in infections remains a substantial global health challenge, contributing to significant morbidity and mortality worldwide in addition to the resistance problem. Five leading bacterial species—Staphylococcus aureus, Escherichia coli, Streptococcus pneumoniae, Klebsiella pneumoniae, and Pseudomonas aeruginosa—accounted for 54.9% of deaths among the bacterial infections investigated.

Current pathogen detection methods, while effective, often rely on slow culture-based techniques that are time-consuming, labor-intensive, and require specialized laboratory facilities. Alternative molecular methods, such as polymerase chain reaction (PCR), offer faster results but still face limitations in accessibility and cost, especially in resource-limited settings. Therefore, there is an urgent need for innovative solutions that can provide real-time, non-invasive, portable/wearable systems for detection of pathogens, enabling clinicians to detect pathogens prior to clinical signs and symptoms of infection, resulting in timely decisions and improve patient outcomes.

### SUMMARY

The present disclosure relates to systems and methods for pathogen detection and wound infection monitoring. The system contains a sensor device configured to detect pathogens by analyzing gaseous emissions from microorganisms that are present in the wound. The system further includes Negative Pressure Wound Therapy (NPWT) or wound dressings to enhance pathogen detection, facilitate early infection identification, and to optimize treatment outcomes.

The system provides real-time monitoring of the metabolically active pathogens via their produced gaseous chemical evidence without the need for invasive sample processing or wound exposure.

The system utilizes advanced Artificial Intelligence (AI) algorithms, including but not limited to machine learning, deep learning, neural networks, and predictive analytics, to detect and predict infections based on real-time data inputs. These AI techniques may involve supervised learning, unsupervised learning, reinforcement learning, and other data-driven methodologies that enhance the system's ability to identify patterns indicative of pathogen presence and infection risk. By analyzing a continuous stream of sensor data, the algorithms provide precise, timely insights that assist clinicians in making informed decisions regarding infection management.

The sensor device has a sensor array that responds to more or more compounds in the gas phase, including but not limited to volatile organic compounds (VOCs), sulfur-containing compounds, and nitrogen-containing compounds (e.g. ammonia), carbon dioxide (CO₂). These molecules are metabolic byproducts of pathogens such as bacteria and fungi, and their presence in the wound environment is indicative of microbial activity.

The sensor array may include one or more types of gas sensors, including but not limited to resistive, capacitive, electrochemical, optical, impedance, photoionization, field-effect transistor (FET) sensors, and surface plasmon resonance (SPR), allowing detection of one or more chemical compounds.

The resistive gas sensors are chemiresistive electronics made from a range of materials, including but not limited to metal oxide (MOS), carbon-based (e.g., graphene and carbon nanotubes (CNTs)), polymers, nanomaterials, composite materials, silicon-based materials, noble metals (e.g., gold, platinum), and transition metal dichalcogenides (TMDs) materials. Each material provides unique responses answering to their properties. The sensors are screened based on criteria such as enhanced sensitivity, selectivity, stability or robustness, tailored for detecting gases emitted by pathogenic organisms. The sensor is calibrated to detect gases at concentrations ranging from 10 ppb to 1000 ppm, typically emitted by pathogenic organisms, which may indicate infection or colonization in a wound environment.

One embodiment of the sensor device has an array of sensor chips. Each sensor chip is mounted on a substrate (e.g., a PCB board) and are connected to electrodes to form an electrical conduit. The electrodes interact with the gaseous emissions, detecting changes in electrical properties that are indicative of the presence of pathogens. The substrate serves to stabilize the electrodes and ensure reliable signal transmission for accurate detection. The use of multiple sensors allows for the detection of a variety of chemical compounds in the gas phase, improving the system's ability to identify various pathogens based on their unique gas signatures. By analyzing the combination of gases emitted by metabolically active pathogens, the sensor array enhances the accuracy of early infection detection.

The sensor device may further include a gas chamber with microfluidic channels. The microfluidic channels that regulate the movement of gases within the chamber, allowing for optimal exposure of the sensor array to the gas.

In some embodiments, the sensor device includes components for real-time pressure monitoring. The sensor device also has algorithm for exclude outlier, e.g., synchronized data denial capabilities, and selectively transmit measurements from a stabilized period.

The sensor device further includes read-out circuits, a microcontroller, a digital signal processing circuit (DSC), analog-to-digital converters (ADC), communication interfaces (e.g., USB, Bluetooth, and WIFI).

The controller includes a microcontroller (MCU) with an integrated ADC, a serial input/output interface, and a wireless communication interface. The output voltage signal of the read-out circuit is required to transmit the measured data to user software within 0.1 second to 24 hours of the test gas being detected on nanosensors. ADC is for data acquisition and digitalization. The communication interface is responsible to set up a bi-directional communication channel with the user software through the Bluetooth or BLE channel.

The MCU includes an executable program stored thereon. The executable program may include Artificial Intelligence (AI) algorithms to analyze data and interpret the gas phase chemical fingerprint. Data from the gas sensor array is processed by MCU, which identifies pathogen signatures, quantifies microbial load, and determines infection risk in real-time.

The sensor device is powered by a battery. Based on the power load, the lifespan of the battery can be in the range of 1-720 days. This extended operational timeframe ensures real-time monitoring during critical stages of wound healing. The sensor device may also have a power cord for connection with an external power source. The power supply powers all sensor and communication functions with versatile solutions of power supplies, making the device suitable for both in-patient and at-home care environments.

The sensor device is equipped with components for wireless and/or wired communications. The wireless communication components include but are not limited to Bluetooth, Wi-Fi, Zigbee, LoRa, 4G/5G, NFC, RFID, and NB-IoT, allowing for real-time data transmission and remote monitoring in various healthcare environments. They may transmit sensor data to an external central monitoring and recording system, e.g., an Electronic Medical Record (EMR) system. Data may be related to wound status updates, pathogen identification, antibiotic efficacy tracking, and alerts when predefined thresholds, such as bacterial load or infection risk, are exceeded, enabling timely clinical intervention. This wireless functionality allows healthcare providers to monitor the wound condition, receive real-time updates on gas concentration levels, and promptly adjust treatment protocols based on the transmitted data.

Some embodiments of the sensor device further include one or more visual indicators, e.g., an LED light, to visually communicate the wound's condition to healthcare providers. For example, green light may indicate that the power is on. Red light may suggest that infections have been detected, prompting immediate medical attention. Different blinking modes can be programmed for various pathogens. Further a yellow light suggests that the device is undergoing baseline calibration or is in a warming-up initialization process, as well as other conditioning and device self-diagnosis modes. Blinking may be used to indicate malfunction of the device itself.

The wound monitoring system further includes one or more NPWT dressings and one or more NPWT pumps. The sensor device is connected in-line with the NPWT dressing to the NPWT pump. This placement enables real-time gas sampling from the wound exudates while maintaining the sub-atmospheric pressure necessary for NPWT to facilitate wound healing.

In another embodiment of the wound monitoring system, the sensor device may be attached to standard wound dressings with additional accessories for gas sampling, enabling a broad application in various clinical and non-clinical settings.

In one embodiment, the sensor device includes a sensor array configured to detect a gaseous emission from one or more pathogens present in a wound by generating signals upon exposure to one or more compounds in the gas phase, the one or more compounds being selected from volatile organic compounds (VOCs), carbon dioxide (CO₂), ammonia (NH₃), sulfur-containing compounds, and nitrogen-containing compounds; a microcontroller (MCU) operatively coupled with the sensor array, the MCU configured to process signals from the sensor array to identify the one or more compounds; a wireless communication means for transmitting data from the MCU to one or more external devices; and a visual feedback means comprising one or more visual indicators that presents information comprising an operational mode of the sensor device and a presence of pathogen in the gaseous emission.

According to one aspect of the embodiment, the sensor array includes a plurality of sensors selected from one or more capacitive sensors, resistive sensors, electrochemical sensors, optical sensors, and field-effect transistor sensors, and surface plasmon resonance (SPR) sensors, each configured to detect chemical changes in the wound environment.

According to another aspect of the embodiment, each visual indicator is operatively connected to the MCU, and is selected from an LED light, an LCD display, an LED display, and an electronic ink display. The display presents information that includes a pathogen type, and a severity of pathogen load, and monomicrobial or polymicrobial status.

According to still another aspect of the embodiment, the MCU is configured to store baseline data of gaseous emissions from the wound and to compare signals from the sensor array to the baseline data to detect deviations indicative of a pathogenic activity.

According to a further aspect, pathogens detected by the sensor device include bacterial species or fungal such as Enterococcus faecium, Staphylococcus aureus (including Methicillin-resistant Staphylococcus aureus or MRSA), Klebsiella pneumoniae, Acinetobacter baumannii, Pseudomonas aeruginosa, Enterobacter species, Escherichia coli, and fungal species including Aspergillus Species, Candida Species, Fusarium Species, Mucorales (Zygomycetes), Scedosporium Species, Curvularia Species, Alternaria Species, Trichophyton Species, Exophiala Species, Cladosporium Species, Bipolaris Species, Penicillium Species, and Phialophora and Fonsecaea Species.

In another embodiment of the current disclosure, a wound monitoring system includes the sensor device connected to a Negative Pressure Wound Therapy (NPWT) dressing and an NPWT pump. Optionally, a gas-liquid separator connected between the NPWT dressing and the sensor device.

According to still a further embodiment, the method for detecting and monitoring infection in a wound includes the steps of applying the NPWT dressing to a wound in a subject in need thereof; starting the NPWT pump to establish a gas flow from the NPWT dressing to the NPWT pump through the sensor device; obtaining a baseline value of the pathogen present in the gaseous emission using the sensor device; monitoring a change in the pathogen level present in the gaseous emission over a period of time using the sensor device; and transmitting information to the visual indicator or an external device, wherein the information comprises the operational mode of the sensor device and the presence of pathogen in the gaseous emission.

The system and method of the present disclosure can be applied to seat cushions for wheelchairs or chairs, hospital beds, mattress toppers or overlays, and wearable devices, providing a personalized and effective monitoring solution for pressure ulcer prevention.

### BRIEF DESCRIPTION OF THE DRAWINGS

The teachings of the present invention can be readily understood by considering the following detailed description in conjunction with the accompanying drawings.

Throughout the drawings and the detailed description, unless otherwise described, the same drawing reference numerals will be understood to refer to the same elements, features, and structures. The relative size and depiction of these elements may be exaggerated for clarity, illustration, and convenience.

FIG. 1 is a schematic illustration of an embodiment of the wound monitoring system of the current disclosure.

FIG. 2 illustrates the deployment of the wound monitoring system.

FIG. 3A is an isometric view of a sensor device of the current disclosure.

FIG. 3B is a section view of the sensor device in FIG. 3A.

FIG. 4 illustrates top view of one embodiment of the sensor device.

### DETAILED DESCRIPTION

The following detailed description, in conjunction with the accompanying drawings, provides a more complete understanding of the disclosure and its various embodiments. The description is not intended to be limiting, and modifications and variations within the scope of the disclosure will be apparent to those skilled in the art.

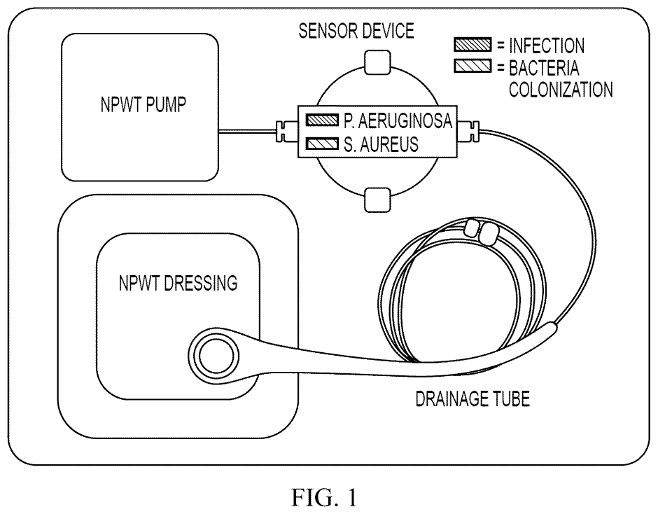

FIG. 1 illustrates an embodiment of the infection monitoring system of the current disclosure. They infection monitoring system includes a vacuum pump (i.e., NPWT pump), an NPWT dressing that can form a seal around a wound, and a sensor device. The NPWT dressing is connected to the sensor through a drainage tube, which in turn is connected to the NPWT pump.

The NPWT dressing has a porous foam made of polyurethane or polyvinyl alcohol. The NPWT pump, once turned on, may create a negative pressure in the range of −40 mmHg to −200 mmHg, e.g., −125 mmHg. Volatile organic compounds (VOC) emitting from the wound flow with the gas through the drainage tube through the sensor device to the vacuum pump. The sensor device contains an array of sensor that can detect one or more VOCs, thereby converting the exposure to VOCs into electric signals. The electric signals obtained from the sensor array is matched with known electric signature patterns of VOCs for identification. In FIG. 1, the sensor device is equipped with a sensor array that is sensitive to Pseudomonas aeruginosa (P. aeruginosa) and Staphylococcus aureus (S. aureus). The pump operates intermittently or continuously, based on the need of wound management. By reading the change of VOCs representative of certain infections, one may monitor the healing of the wound.

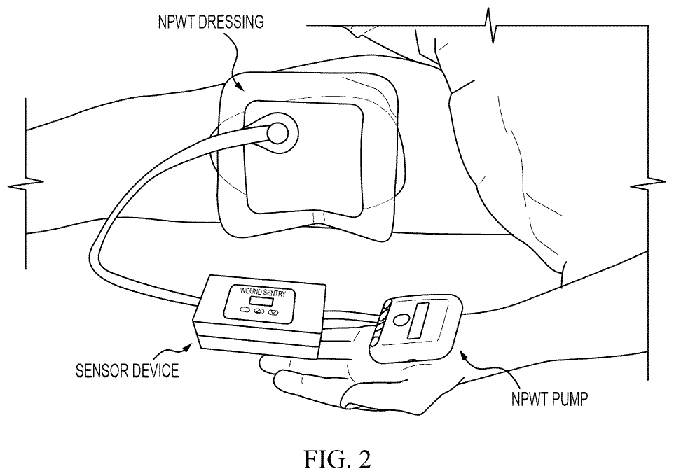

FIG. 2 shows the deployment of the infection monitoring system. The NPWT dressing is applied around a wound on a patient's thigh. The sensor device has an inlet and an outlet. The inlet of the sensor device is connected to the NPWT dressing through a tube and the outlet is connected to the NPWT pump through a tube. The size of the NPWT dressing as well as the pressure setting of the NPWT pump are determined according to the wound management needs.

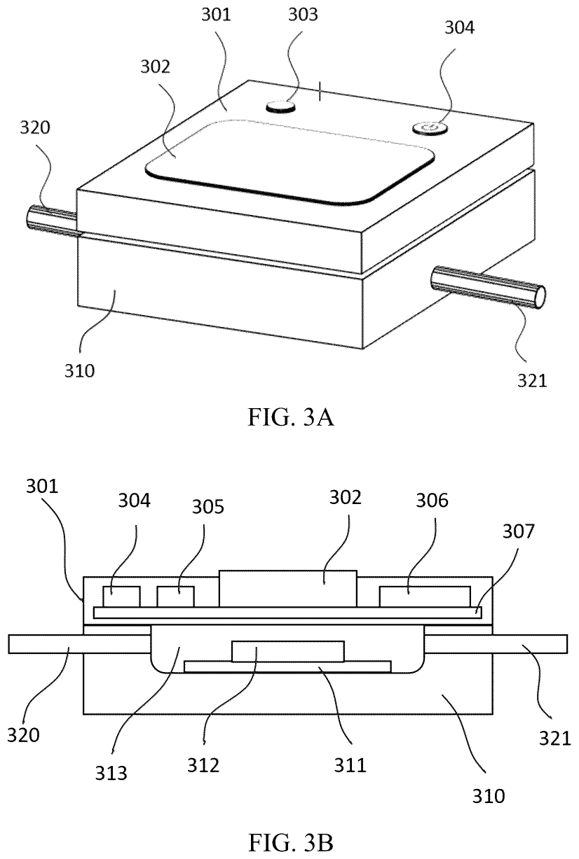

FIG. 3A and FIG. 3B show the details of the sensor device. As shown in FIG. 3A, the sensor device is in the shape of a box having a tope cover 301 and a bottom case 310. An inlet 320 and an outlet 321 are installed on the bottom case 310. The top cover has a display 302, an LED light 303, and a power button 304 installed thereon.

FIG. 3B shows some of the components inside the sensor device. The bottom case 310 contains a well 313, to which the inlet 320 and the outlet 321 extend into. The sensor array 312 contains a plurality of sensor elements configured to detect one or more of VOCs, CO₂, NH₃, and other pathogen-related gases. The sensor array 312 is installed on the PCB 311. The sensor array 312 and the PCB 311 are disposed in the well 313.

The top cover 301 contains one or more substrates 307. The battery 306, the display 302, the MCU 305, and the data transfer unit 304 are disposed on the one or more substrates 307. The top cover 301 closes the well 313. The enclosed well 313 serves as a gas chamber during operation.

In some embodiments, the sensor element is a sensor chip and electrodes it is connected to, which constitutes the basic unit of the sensor array. Each sensor array contains plural sensor chips connected to electrodes. The sensor chip has a sensing material, when exposed to certain compounds in the gas, generates electric signals in response. Higher concentrations of the certain compound generate stronger signals. Accordingly, the sensor element can respond the presence as well as the concentration of certain compound.

In other embodiments, sensor elements in the sensor array are sensitive to different compounds so that the sensor array is sensitive to multiple compounds.

The MCU 305 is an embedded device containing an executable program that is configured to analyze electrical signals received from the sensor array 312. The data transfer unit 304 is connected to the MCU 305. It may contain wireless communication device such as a Bluetooth device or a wi-fi device. The data transfer unit 304 may also be a wired communication device that connects to a PC through an Ethernet cable or a USB connection.

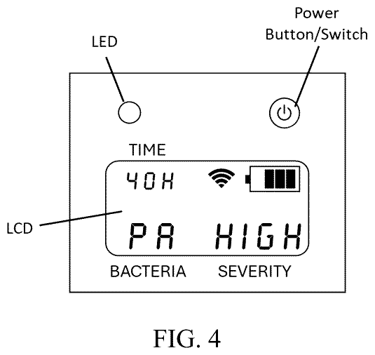

The display 302 can be an LED display or an LCD display. As illustrated in FIG. 4, it displays information such as the operational time (e.g., “40 h”), the bacteria being detected (e.g., “PA” or Pseudomonas aeruginosa), infection severity (e.g., “HIGH,” “MEDIUM,” or “LOW”), the system connectivity by showing Wi-Fi/Bluetooth icon, and a battery indicator shows the device's power status.

The wound monitoring system can be operated according to a method described below. First, the dressing is applied to the wound and the NPWT pump is powered on to reduce the pressure in the wound monitoring system.

The pressure sensor in the wound monitoring system, e.g., in the sensor device, in the NPWT pump, in the NPWT dressing, or in the tube. Once the wound monitoring system reaches a preset target pressure, the sensor device enters a baseline calibration mode, during which the sensor array measures the initial pathogen level in the wound environment to establish a baseline. This calibration process typically may take tens of seconds to several minutes, during which time the yellow LED is illuminated. Once calibration is complete, the yellow LED is off, leaves only green LED on, signaling that the device is ready for continuous real-time monitoring.

During operation, the sensor device continuously samples the gas flowing through the sensor device, acquiring the chemical features of components in the gas sample, which can be a combination of VOCs, CO₂, NH₃, and other metabolic byproducts associated with pathogen activity. The MCU processes this data and applies AI algorithms to detect infection patterns.

The pathogens of wound infection can be one or more bacterial or fungi species, such as Acinetobacter anitratus, Acinetobacter baumannii, Actinomyces israelii, Agrobacterium radiobacter, Agrobacterium tumefaciens, Anaplasma phagocytophilum, Aspergillus fumigatus, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Candida albicans, Helicobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Candida species, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus gallinarum, Enterococcus maloratus, Escherichia coli, Francisella tularensis, Fusobacterium nucleatum, Enterobacter species, Fusarium solani, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactobacillus delbrueckii, Lactococcus lactis, Legionella pneumophila, Listeria monocytogenes, Methylobacterium extorquens, Microbacterium multiforme, Micrococcus luteus, Moraxella catarrhalis, Morganella morganii, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexicoense, Neisseria gonorrhoeae, Neisseria meningitidis, Pasteurella multocida, Pasteurella tularensis, Porphyromonas gingivalis, Prevotella melaninogenica, Proteus vulgaris, Proteus mirabilis, Proteus penneri, Providencia stuartii, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faecium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Yersinia enterocolitica, Yersinia pestis and Yersinia pseudotuberculosis, and/or known to include one or more antibiotic-resistant strains descending from a known species, and/or known to comprise one or more extended spectrum beta-lactamase-producing strains descending from a known species, in particular the one or more extended spectrum beta-lactamase-producing strain is selected from the group consisting of: extended spectrum beta-lactamase-producing Escherichia coli, and extended spectrum beta-lactamase-producing Klebsiella pneumoniae. The fungal species include but not limited to Aspergillus Species, Candida Species, Fusarium Species, Mucorales (Zygomycetes), Scedosporium Species, Curvularia Species, Alternaria Species, Trichophyton Species, Exophiala Species, Cladosporium Species, Bipolaris Species, Penicillium Species, Phialophora and Fonsecaea Species (Agents of Chromoblastomycosis).

The Antibiotic-resistant bacterial strains may include Carbapenem-resistant Acinetobacter baumannii, carbapenem-resistant Pseudomonas aeruginosa, vancomycin-resistant Enterococcus faecium, methicillin-resistant Staphylococcus aureus, vancomycin-resistant Staphylococcus aureus, clarithromycin-resistant Helicobacter pylori, fluoroquinolone-resistant Campylobacter coli, fluoroquinolone-resistant Campylobacter fetus, fluoroquinolone-resistant Campylobacter jejuni, fluoroquinolone-resistant Helicobacter pylori, fluoroquinolone-resistant Salmonella enteritidis, fluoroquinolone-resistant Salmonella typhi, fluoroquinolone-resistant Salmonella typhimurium, cephalosporin-resistant Neisseria gonorrhoeae, fluoroquinolone-resistant Neisseria gonorrhoeae, penicillin-non-susceptible Streptococcus pneumoniae, ampicillin-resistant Haemophilus influenzae, fluoroquinolone-resistant Shigella dysenteriae, carbapenem-resistant Escherichia coli, carbapenem-resistant Klebsiella pneumoniae, carbapenem-resistant Enterobacter cloacae, carbapenem-resistant Serratia marcescens, carbapenem-resistant Proteus vulgaris, carbapenem-resistant Proteus mirabilis, carbapenem-resistant Proteus penneri, carbapenem-resistant Providencia stuartii, carbapenem-resistant Morganella morganii, cephalosporin-resistant Escherichia coli, cephalosporin-resistant Klebsiella pneumoniae, cephalosporin-resistant Enterobacter cloacae, cephalosporin-resistant Serratia marcescens, cephalosporin-resistant Proteus vulgaris, cephalosporin-resistant Proteus mirabilis, cephalosporin-resistant Proteus penneri, cephalosporin-resistant Providencia stuartii and cephalosporin-resistant Morganella morganii.

When the sensor device identifies gas patterns indicative of infection, the red LED is illuminated, and an alert is sent to the connected EMR system via the wireless communication interface. This enables healthcare providers to take timely action, review microbial activity trends, and assess pathogen load. The data can be accessed remotely through the hospital's EMR platform, ensuring that physicians have real-time access to wound status and the effectiveness of therapeutic interventions.

After an infection has been identified and treated, the device continues to monitor gas emissions for pathogen fingerprints. The sensor device can be reset to tare mode for recalibration if necessary, allowing for continuous surveillance of bacterial load and wound healing progress. This feature ensures that healthcare providers can track the trajectory of the wound's recovery and adjust treatments as needed.

The sensor device may operate continuously or intermittently at a certain interval, e.g., one or more tests per day. Each test may take several minutes. During idle periods, the device enters a low-power mode to conserve energy. The data acquisition process is coordinated with the pumping cycles, and the data is processed based on the designated pressure range.

The sensor device can optionally be equipped with sampling loops enclosed by automatic switching valves and shutters to enhance the accuracy of quantitative measurements. During each testing cycle, the flow path is opened automatically, directing the gas to the gas chamber containing the sensor array. After testing for a preset duration (e.g., 3 minutes) and the stabilization of the test data, the valve or shutter on side of the inlet to the sensor device is turned off. The sensor device is still connected to the pump so that the gas in the gas chamber can be evacuated.

The monitoring system may further include a gas-liquid separator connected between the NPWT dressing and the sensor device to remove the liquid from the gas flow.

While the present disclosure has been described with reference to specific embodiments, it will be understood by those skilled in the art that various changes may be made, and equivalents may be substituted without departing from the scope of the disclosure. In addition, many modifications may be made to adapt a particular situation or material to the teachings of the disclosure without departing from the scope thereof. Therefore, it is intended that the present disclosure is not limited to the particular embodiments disclosed, but that the disclosure will include all embodiments falling within the scope of the appended claims.

We claim:

1. A sensor device, comprising:
a sensor array configured to detect a gaseous emission from one or more pathogens present in a wound by generating signals upon exposure to one or more compounds in the gas phase, the one or more compounds being selected from volatile organic compounds (VOCs), carbon dioxide (CO₂), ammonia (NH₃), sulfur-containing compounds, and nitrogen-containing compounds;
a microcontroller (MCU) operatively coupled with the sensor array, the MCU configured to process signals from the sensor array to identify the one or more compounds;
a wireless communication means for transmitting data from the MCU to one or more external devices; and
a visual feedback means comprising one or more visual indicators that presents information comprising an operational mode of the sensor device and a presence or absence of pathogen in the gaseous emission.

2. The sensor device of claim 1, wherein the sensor array comprises a plurality of sensors, each sensor is selected from one or more capacitive sensors, resistive sensors, electrochemical sensors, optical sensors, and field-effect transistor sensors, and surface plasmon resonance (SPR) sensors, and is configured to detect the one or more compounds in the wound environment.

3. The sensor device of claim 1, wherein each visual indicator operatively connected to the MCU, and is selected from an LED light, an LCD display, an LED display, and an electronic ink display, and further displays information comprising a pathogen type, and a severity of pathogen load, and monomicrobial or polymicrobial status.

4. The sensor device of claim 1, wherein the MCU is configured to store baseline data of the gaseous emission from the wound and to compare signals from the sensor array to the baseline data to detect deviations indicative of a pathogenic activity.

5. The sensor device of claim 1, wherein the wireless communication means is configured to transmit data to the one or more external devices when a concentration of pathogen in the gaseous emission exceeds a predefined threshold.

6. The sensor device of claim 1, wherein the pathogens include bacterial species and/or fungal selected from Enterococcus faecium, Staphylococcus aureus (including Methicillin-resistant Staphylococcus aureus or MRSA), Klebsiella pneumoniae, Acinetobacter baumannii, Pseudomonas aeruginosa, Enterobacter species, Escherichia coli, and fungal species including Aspergillus Species, Candida Species, Fusarium Species, Mucorales (Zygomycetes), Scedosporium Species, Curvularia Species, Alternaria Species, Trichophyton Species, Exophiala Species, Cladosporium Species, Bipolaris Species, Penicillium Species, and Phialophora and Fonsecaea Species.

7. A wound monitoring system, comprising the sensor device of claim 1, a Negative Pressure Wound Therapy (NPWT) dressing, and an NPWT pump, wherein the sensor device is disposed between and connected to the NPWT dressing and the NPWT pump.

8. The wound monitoring system of claim 7, wherein the sensor device having an inlet connected to the NPWT dressing and an outlet connected to the NPWT pump.

9. The wound monitoring system of claim 8, further comprises a gas-liquid separator connected between the NPWT dressing and the inlet of the sensor device.

10. A method for detecting and monitoring infection in a wound using the wound monitoring system of claim 7, comprising:
applying the NPWT dressing to a wound in a subject in need thereof;
starting the NPWT pump to establish a gas flow from the NPWT dressing to the NPWT pump through the sensor device;
obtaining a baseline value of the pathogen present in the gaseous emission using the sensor device;
monitoring a change in the pathogen level present in the gaseous emission over a period of time using the sensor device; and
transmitting information to the visual indicator or an external device, wherein the information comprises the operational mode of the sensor device and the presence or absence of pathogen in the gaseous emission.

11. The method of claim 10, wherein the NPWT pump generates a negative pressure in the range of −40 mmHg to −200 mmHg in the wound monitoring system.

12. The method of claim 10, wherein the sensor array is configured to detect a pathogen concentration in the gaseous emission in the range of 0.01 ppm to 1000 ppm.
